(19) United States
(12) Patent Application Publication
(10) Pub. No.: US 2020/0185071 A1
(43) Pub. Date: Jun. 11, 2020

(54) FACILITATING SEXUALLY TRANSMITTED INFECTION SERVICES

(71) Applicant: Binx Health Limited, Trowbridge (GB)

(72) Inventor: Jeffrey Luber, Cambridge, MA (US)

(21) Appl. No.: 16/707,721

(22) Filed: Dec. 9, 2019

Related U.S. Application Data
(60) Provisional application No. 62/833,358, filed on Apr. 12, 2019.
(60) Provisional application No. 62/777,561, filed on Dec. 10, 2018.

(51) Int. Cl. G16H 10/60; G06F 21/32; G16H 15/00; G16H 10/20; G16H 50/80; C12N 15/113

(52) U.S. Cl. CPC G16H 10/60; G06F 21/32; C12N 15/1132; G16H 10/20; G16H 50/80; G16H 15/00

(57) ABSTRACT

Systems and methods to facilitate services related to sexually transmitted disease (STI) testing and therapy are described herein. The invention relates to secure systems, methods, and devices for receiving, generating and managing user health data, creating user-specific profiles, and controls to facilitate requisition and delivery of services and products related to diagnostic testing and therapy. User-specific devices may be used to facilitate STI services described herein and may be used to provide information to a user and to collect information about a user.

### RELATED APPLICATIONS

The application claims priority to and benefit of U.S. Provisional Patent Application No. 62/833,358, filed Apr. 12, 2019 and U.S. Provisional Patent Application No. 62/777,561 filed Dec. 10, 2018, the contents of each of which are incorporated by reference herein in their entireties.

### FIELD OF INVENTION

The invention relates to systems and methods for disease diagnostics and testing.

### BACKGROUND

More than one million sexually transmitted infections (STIs) are acquired worldwide, every day. See, World Health Organization (WHO) Website. Report on global sexually transmitted infection surveillance, 2015. Worldwide it is estimated that there are 357 million new cases of four of the curable STIs (i.e., Chlamydia trachomatis, Neisseria gonorrhoeae, syphilis, and Trichomonas vaginalis) among 15-49 year olds. Id. In 2017, nearly 2.3 million cases of chlamydia, gonorrhea, and syphilis were diagnosed in the United States alone. See, Centers for Disease Control and Prevention (CDC) Website. Press Release: New CDC analysis shows steep and sustained increases in STDs in recent years, Atlanta, Ga.: Centers for Disease Control, 2018.

The prevalence of viral STIs is striking, with an estimated 417 million people infected with herpes simplex type 2, and approximately 291 million women harboring the human papillomavirus (HPV) worldwide. See, WHO Website. Report on global sexually transmitted infection surveillance, 2015. According to the CDC, 79 million Americans, mostly in their late teens and early 20s, are infected with HPV, making it the most common STI in the US. See, CDC Website, Press Release: New study shows HPV vaccine helping lower HPV infection rates in teen girls, Atlanta, Ga.: Centers for Disease Control 2013.

Significantly, an estimated 1,122,900 people were living with the human immunodeficiency virus (HIV) at the end of 2015, 150% of whom had yet to receive a diagnosis. See, CDC Website. HIV Surveillance Supplemental Report 2018;23(1). These numbers are significant despite the recent decline of HIV infection in the US and increased HIV prevention efforts, including pre-exposure prophylaxis (PrEP).

PrEP is when people are at risk for HIV infection take daily HIV medications (i.e., antivirals) to lower their chances of becoming infected with HIV. Similar medications can also be taken daily to control the spread of HIV infection or decrease HIV-related illnesses. Though highly effective when taken as prescribed, compliance with the prescribed protocols is crucial for these drugs to be effective. Patients on a PrEP protocol must also submit to quarterly HIV testing to ensure compliance and minimization of complications.

Despite the systems in place to educate people about STIs, prevent them, and treat them, STIs are still on the rise. Whether it is the societal stigmas or the lack of healthcare resources, people are simply not getting tested for STIs after having unprotected sex. This STI epidemic is not only associated with high financial costs, but also with other health-related costs, such as adverse reproductive outcomes and increased vulnerability to other infections and diseases. In a time of total social transparency, discussion of STIs and STI testing remain a taboo topic, scarcely discussed even between physician and patient.

### SUMMARY

The invention solves for problems associated with accessibility and anonymity of STI testing and therapy. Instead of going to a physician's office or a clinic for STI testing, methods and systems of the invention facilitate secure ordering and delivery of diagnostic test kits and therapeutics directly to a user via a user's interaction with a device. The device is user-specific and has an interactive component, such as a button, switch, touch-screen, or biometric sensor, which is integrated with processing software.

The interactive component of the user-specific device initiates a command when the user interacts with the device by, for example, pressing a button. The user-specific device is connected to a database and processing software relating to the patient's medical history, prescribing information, claims data, address, payment information and other personal information, including passwords and biometric templates. The user-specific device may be placed in the user's bag, a drawer, fashioned to be attached to a key chain, or attached to a hard surface by a magnet or adhesive and may be inconspicuously designed to protect the privacy of the user. The user-specific device requires a form of authentication, such as password verification, biometric identification, or multi-factor authentication to verify the user's request via the user-specific device. Authentication may be via the interactive component. For example, the user enters a password via the touch-screen on the device. Similarly, the user may enter biometric data, such as the user's fingerprint, via the biometric sensor on the user-specific device. The server authenticates the user by comparing the user's entry to the information previously entered and stored in the user's profile. In other embodiments of the invention, the user-specific device may also initiate multi-factor authentication when the user interacts with the device.

The user-specific device is configured to communicate with a server, for example communicating requests for STI diagnostic tests initiated by the user's interaction with the device. The server stores user profiles, including the user's authentication information, and associates a user's profile with the user-specific device. A user's authentication information can be a password or a biometric template. In some embodiments of the invention, upon receipt of a request, the server authenticates the user by performing one or more authentication process, including but not limited to biometric identification, password validation, and multi-factor authentication. The server may authenticate the user and authorize the request by comparing information entered with the request to the authentication information of the user or by multi-factor authentication. Multi-factor identification includes the server receiving a communication from the user-specific device, generating and communicating an authentication factor to the user's computer or mobile device, receiving at least a portion of the authentication factor from the user's computer or mobile device, authenticating the user by processing the authentication factor received from the user. Once authenticated, the request is authorized and the server processes the request based on commands associated with the user's profile, such commands may include delivery of a diagnostic test kit or a therapeutic to the user. Particularly, the package delivered to the user is inconspicuously labeled so as to maintain the user's privacy by not indicating the contents of the package or the source. As such, the systems and methods of the invention overcome the issues associated with diagnostic services, such as STI related services and therapies, by providing accessible and private testing and therapy options to minimize the spread of STIs.

Accordingly, the invention provides systems and methods for receiving, generating and managing user data. Systems and methods of the invention manage user data in a database, such as a user health management database or platform. User-specific profiles may be created by systems and methods described herein. User profiles may be used to input data or to receive user data or information. User data includes a user's health data, including but not limited to the user's medical records, claims data, diagnoses, diagnostic test requests, test results, physician information, prescription protocols, prescriptions or prescribing information, requests for diagnostic tests, or other personal information, for example, a user's address or payment information. User data also includes authentication information, including biometric templates and passwords. User data is maintained on and by the systems and methods of the invention. In some embodiments, the data may include user-specific device registration data and data associated with the user's interaction with the user-specific device. The platform and user profile may be accessible via a computing device or a mobile application.

Systems and methods of the invention enable a user to select commands. Commands may be identifying a diagnostic test or a prescription to order, scheduling a reoccurring diagnostic test or prescription refill, or setting a reminder to do the same or to take a prescription. A user's commands may be communicated to a user-specific device. Commands available to a user may be based on user data, for example, a user's diagnosis, prescription information, additional input from the user, data from a physician or a pharmacy, or data from a user-specific device. In other embodiments, the commands may be generated by systems and methods of the invention and may be user-specific. In yet other embodiments, the commands generated by the systems and methods and are provided to the user and the user may accept, edit or delete the commands.

Systems and methods of the invention maintain commands. For example, commands are maintained by a server, and when the user interacts with its user-specific device, the server may generate a corresponding command. For example, a user may set a command to request an HIV diagnostic test when the user interacts with a user-specific device. The user may then interact with the device to initiate the command, for example, the ordering of an HIV test. In some embodiments, user interaction may be pressing a button on the device, which initiates the command. In some embodiments, the systems and methods described herein may perform the same command each time the user interacts with the device, via the server.

In addition to receiving user requests, systems and methods disclosed herein may generate information to the user. For example, prompts may be generated to remind a user to order a diagnostic test, a prescription, or to comply with a prescription protocol. A prompt may be generated based on the user profile, user commands, or by a communication received from a third party, such as a physician or pharmacy. A prompt may be delivered to the user via the user-specific device and may be in the form of an audio, physical, or visual signal generated by the device. For example, a command may be set by the user to remind the user to order an STI diagnostic test. The user-specific device may then generate a signal, such as a vibration, light, or sound to remind or prompt the user to order the test. In some embodiments, prompts are reoccurring.

Systems and methods of the invention may also track and report shipment status and delivery status of the diagnostic test kit or prescription, status of a returned kit containing the user's specimen for testing, and the diagnostic testing stage of the sample. In embodiments of the invention, diagnostic testing results are returned to a user and maintained by the platform and are accessible to the user by entering the user's profile. Systems and methods of the invention may use test results to prompt a user to order a diagnostic test. In other embodiments, a therapy or prescription may be recommended or prescribed.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a block diagram illustrating one embodiment of an exemplary system for facilitating STI services.

FIG. 2 is a block diagram illustrating the user health management platform described in FIG. 1 in greater detail.

FIG. 3 is a flow diagram illustrating user input of information into the user health management platform.

FIG. 4A is a block diagram illustrating at least one embodiment of an exemplary user-specific device with a button to facilitate STI services, such as ordering an STI diagnostic test kit when a user presses the button.

FIG. 4B is a block diagram illustrating at least one other embodiment of an exemplary user specific device that generates a signal to facilitate STI services, such as a reminder or prompt via a signal.

FIG. 5 is a block diagram illustrating at least one embodiment the system for facilitating STI services.

FIG. 6 is a block diagram illustrating at least one embodiment of the user-specific device.

FIG. 7A is a block diagram illustrating an example of authentication and authorization of a user's request via a user-specific device.

FIG. 7B is a flow diagram of an example of the method of authentication and authorization of a user's request via a user-specific device.

Features and advantages of the claimed subject matter will be apparent from the following detailed description of embodiments consistent therewith, which description should be considered with reference to the accompanying drawings.

### DETAILED DESCRIPTION

Methods and systems of the present invention include those which facilitate STI services, including STI diagnostic tests and therapies. A user creates a user profile, sets commands, and registers a physical user-specific device to be associated with the user profile and commands, such as an electronic device comprising an interactive component. The interactive component, for example, a button, is interacted with by the user by the user pressing the button when she wants to order the STI test kit to be delivered to her. The package containing the kit is inconspicuously labeled to protect the privacy of the user and may contain a specimen collection device. The user then returns her biological specimen in the specimen collection device to be processed by a laboratory. The results of the STI test may then be delivered to the user via the systems and methods described herein.

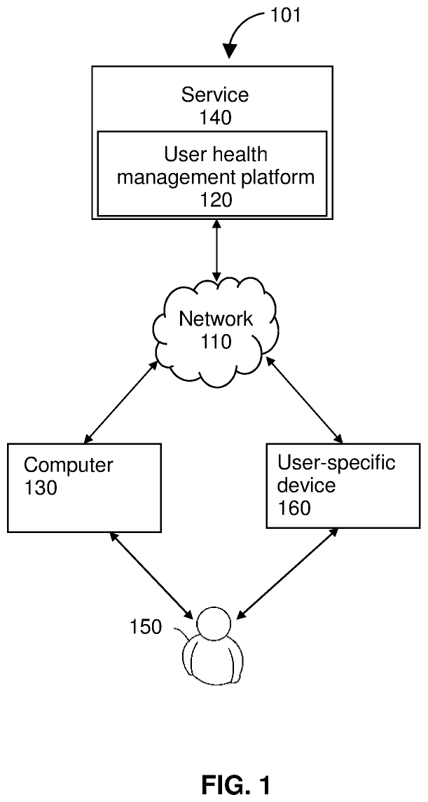

FIG. 1 illustrates one embodiment of an exemplary system 101 consistent with the present disclosure. As shown, system 101 includes a user health management platform 120 embodied on a computing service 140. For example, the user health management platform 120 may be embodied on a service 140 that is cloud based. The user health management platform 120 is configured to generate or create a profile specific to a user 150 using data inputted by a user via a device 130 or via the user 150 interactions with the user-specific device 160, to communicate with the user 150 via a device 130 or a user-specific device 160, and to receive commands from the user 150 via the user-specific device 160 over a network 110. User data may include, for example, a user's medical records, claims data, diagnoses, diagnostic test requests, test results, physician information, prescription protocols, prescriptions or prescribing information, requests for diagnostic tests, payment information or other personal information, such as a user's address, passwords or biometric templates. In some embodiments, data may be received from a third-party, such as a physician or pharmacy.

The network 110 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web).

The network 110 may be any network that carries data. Non-limiting examples of suitable networks that may be used as network 110 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G) cellular-based data communication technologies, Bluetooth radio, Near Field Communication (NFC), the most recently published versions of IEEE 802.11 transmission protocol standards as of October 2018, other networks capable of carrying data, and combinations thereof. In some embodiments, network 110 is chosen from the internet, at least one wireless network, at least one cellular telephone network, and combinations thereof. As such, the network 110 may include any number of additional devices, such as additional computers, routers, and switches, to facilitate communications. In some embodiments, the network 110 may be or include a single network, and in other embodiments the network 110 may be or include a collection of networks.

The user health management platform 120 is configured to receive requests from the user 150 via the user-specific device 160 which is registered or associated with a user 150. In some embodiments, the user 150 physically interacts with the user-specific device 160 to communicate a request or a command to the user health management platform 120 and cloud-based service 140, and/or other computing devices over the network 110. Accordingly, the user-specific device 160 may be embodied as any type of physical electronic device capable of user 150 interaction. For example, the user-specific device 160 may have an interactive component, for example a button, switch, touch-screen, or a biometric sensor, capable of user interaction. User interaction may be the user pressing the button to initiate the command or request when pressed, for example.

The device 130 may be embodied as any type of device for communicating with the user health management platform 120 and cloud-based service 140, and/or other computing devices over the network 110. For example, at least one of the computing devices may be embodied as, without limitation, a computer, a desktop computer, a personal computer (PC), a tablet computer, a laptop computer, a notebook computer, a mobile computing device, a smart phone, a mobile device, a cellular telephone, a handset, a messaging device, a work station, a distributed computing system, a multiprocessor system, a processor-based system, and/or any other computing device configured to store and access data, and/or to execute software and related applications consistent with the present disclosure. In the embodiments described here, the device 130 is generally embodied as a mobile device, a smart phone, cellular telephone, computer, a personal computer (PC), a tablet computer, a laptop computer, a notebook computer, and the like.

As previously mentioned, the user health management platform 120 is configured to receive data from the user 150 via user's device 130 over the network 110 and uses the data to create a user-specific profile. The user health management platform 120 is configured to receive commands from the user 150 via user's device 130 over the network 110 and uses the commands to initiate orders or requests when the user 150 interacts with the user-specific device 160. For example, the user 150 may set commands on the health management platform 120 to identify a diagnostic test or therapeutic to order, to schedule a reoccurring diagnostic test or prescription refill, or to set a reminder, such as to take or fill a prescription. The commands are communicated by the user 150 via the device 130 to the user health management platform 120 and cloud-based service 140 via the network 110. When the user 150 interacts with the user-specific device 160 the corresponding command is generated or processed by the cloud-based service 140 via the network 110. For example, the command may initiate an order for an STI diagnostic test kit to be shipped to the user 150. The STI test kit may be shipped in an inconspicuous container or package (i.e., packaging that does not reveal the contents) to an address associated with the user profile. The kit may contain a specimen collection device to collect a biological specimen from the user 150. The specimen may be any biological sample obtained from the user 150, e.g., blood, urine, cerebrospinal fluid, seminal fluid, saliva, sputum, stool and tissue. The kit may also include a return package for the completed kit to be returned to a laboratory for diagnostic testing or analysis. The return package may be specifically associated with the user 150 and upon shipment, data is received by the user health management platform 120 and cloud-based service 140 via the network 110. The user health management platform 120 and the service 140 via the network 110 receive data related to the status of the shipment, analysis and the test results, and the user 150 is then able to track the same via the user profile accessible by the device 130. Additionally, the user 150 may receive updates via the user-specific device 160.

A user-specific device 160 may be configured to generate prompts to remind the user 150 order a diagnostic test, a prescription, or to comply with prescription protocol. The prompt may be in the form of an audio, physical, or visual signal generated by the user-specific device 160. The user 150 may then interact with the user-specific device 160 to initiate a corresponding command generated by the cloud-based service 140 via the network 110. Similarly, the user-specific device 160 may be configured to generate alerts related to various status updates. The status updates may include the status of a request, shipment of test kit, delivery of a test kit, a prescription is ready for pick-up, status of diagnostic analysis, test results are available, etc.

The user profile, accessible on a computer provides an interactive interface in which the user inputs information and commands, and receives information and data from systems and methods described herein. All information related to the user is accessible and can be displayed via the user profile. Furthermore, the user health management platform 120 allows for customizable access to the data to someone other than the user 150. In particular, selected members of a healthcare network associated with the user, for example, a physician may be provided access to the user profile. For example, a physician associated with the user 150 can review the data and accept or deny requests submitted by the user 150, or can initiate prompts to remind the user to order a test, schedule an appointment, or reminder for an upcoming appointment.

Embodiments of the system 101 of the present disclosure include computer systems, computer operated methods, computer products, systems including computer-readable memory, systems including a processor and a tangible, non-transitory memory configured to communicate with the processor, the tangible, non-transitory memory having stored instructions that, in response to execution by the processor, cause the system to perform steps in accordance with the disclosed principles, systems including non-transitory computer-readable storage medium configured to store instructions that when executed cause a processor to follow a process in accordance with the disclosed principles, etc.

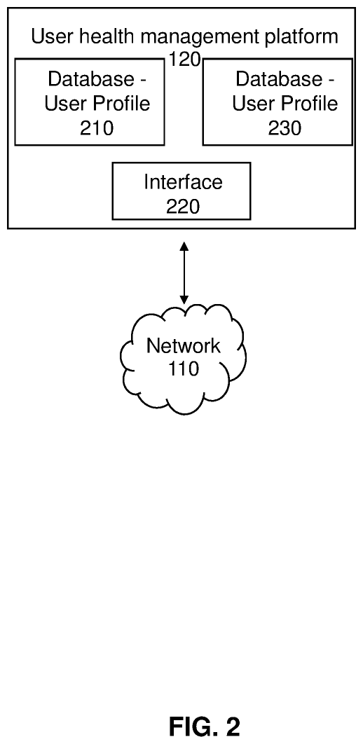

FIG. 2 is a block diagram illustrating the user health management platform of FIG. 1 in greater detail. As shown, the user health management platform 120 may include an interface 220 and various databases for storage of data, such as a user profile 210 for the storage and management of various sets of data, including user data and information, including, but not limited to, health data, medical records, claims data, diagnoses, diagnostic test requests, test results, physician information, prescription protocols, prescriptions information, or requests for diagnostic tests, the data may also include device registration data, authentication information, such as passwords and biometric templates, and data associated with the user's interaction with the user-specific device 160. User data may be stored in various databases on the user health management platform 120. In some embodiments, types of user data (e.g., claims data) may be stored in its own database on the user health management platform 120. In other embodiments, databases for storing and maintaining user profiles and user settings for a given account or a user-specific device database for storing and maintaining data related to any given user-specific device may be on the user health management platform. The user health management platform 120 may be configured to communicate and exchange data with each of the databases.

The interface 220 may generally allow a user to access data or a user profile 210 on the user health management platform 120, via a software application provided on a device 130, such as a mobile device. For example, upon accessing the software application, the interface 220 may be presented to the user 150 via their device 130, in which the user may navigate a dashboard or standard platform interface so as to enter or view data in the user profile, or to assign commands.

The user data collected will be stored on a secure server. User data, such as Personally Identifying Information (PII), passwords, and biometric templates, may be stored in an encrypted form, using symmetric encryption. Particularly, the user-specific device requires a form of authentication, such as password verification, biometric identification, or multi-factor authentication to verify the user's request via the user-specific device. The user may enter a password via the touch-screen on the device, or the user may enter biometric data, such as the user's fingerprint, via the biometric sensor on the user-specific device. The server authenticates the user by comparing the user's input to the information previously entered and stored in the user's profile. For example, passwords may be hashed, and user authentication and authorization of the request may be handled using a server that allows authentication using safe and reliable identity servers. In other embodiments of the invention, the user-specific device may also initiate multi-factor authentication when the user interacts with the device. All PII data may be transported over HTTPS using TLS in its encrypted or hashed forms to be synchronized between the mobile apps and the secure server.

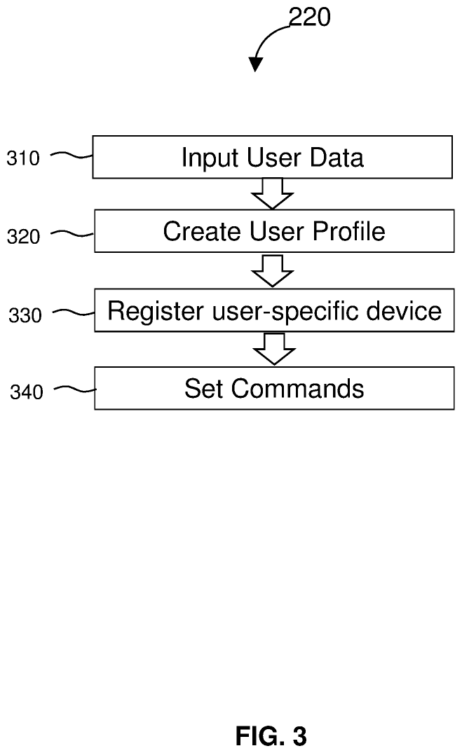

FIG. 3 is flow diagram of how the user 150 may create a user profile 210 and set commands 340 via the interface 220. A user 150 may enter or input their data 310, for example, health related data, and create a user profile 320 on the user health management platform 120. Once the user profile 110 is created, the user can register/associate 330 their user-specific device 160. Registration 330 may include entering a password or a biometric template for authentication purposes. Once registered, the user 150 can set commands 340 such as to identify a diagnostic test or series of tests to order, schedule a reoccurring diagnostic test, or set a reminder to take or fill a prescription.

A user's commands 340 may be communicated to the user-specific device 160. The available commands for a user may be based on the data in the user profile 110, including user's diagnosis, previous tests or prescriptions, input from the user, a physician or a pharmacy, or data collected from the user-specific device. In other embodiments, the commands may be generated by systems and methods of the invention and may be user-specific. In some embodiments, the commands 340 are generated by the user health management platform 120 and service 140 are provided to the user 150. The user 150 may accept, edit or delete the commands 340. In some embodiments, a third party, such as a physician or pharmacy, may generate commands 340 by accessing a user profile 110. The third party may have a separate profile that is associated or linked to the user profile. A third-party profile may also be maintained by systems and methods described herein. In other embodiments, the user may set commands 340 to require authentication of a user or the request before an order can be processed. Authentication may be performed by the server and may include password verification, biometric identification, and/or multi-factor authentication to verify the user's request via the user-specific device. Authentication of the user may be via the interactive component. For example, the user enters a password via the touch-screen on the device. Similarly, the user may enter biometric data, such as the user's fingerprint, via the biometric sensor on the user-specific device. The server authenticates the input by comparing the user's entry to the information previously entered and stored in the user's profile to authenticate the user. In other embodiments of the invention, the user-specific device may also initiate multi-factor authentication when the user interacts with the device.

Test kits for any STI or for compliance with a prescription protocol can be ordered by a user via the systems and methods described herein, including but not limited to diagnostic test kits for activity, bacterial vaginosis, chlamydia, cystitis, gonorrhea, hepatitis A, hepatitis B, hepatitis C, herpes (herpes simplex type 1 and 2), HIV, HPV, lymphogranuloma venereum, molluscum contagiosum, non-gonococcal urethritis, pelvic inflammatory disease, phthirus pubis, syphilis, trichomoniasis, vaginitis, and for compliance with PrEP protocols.

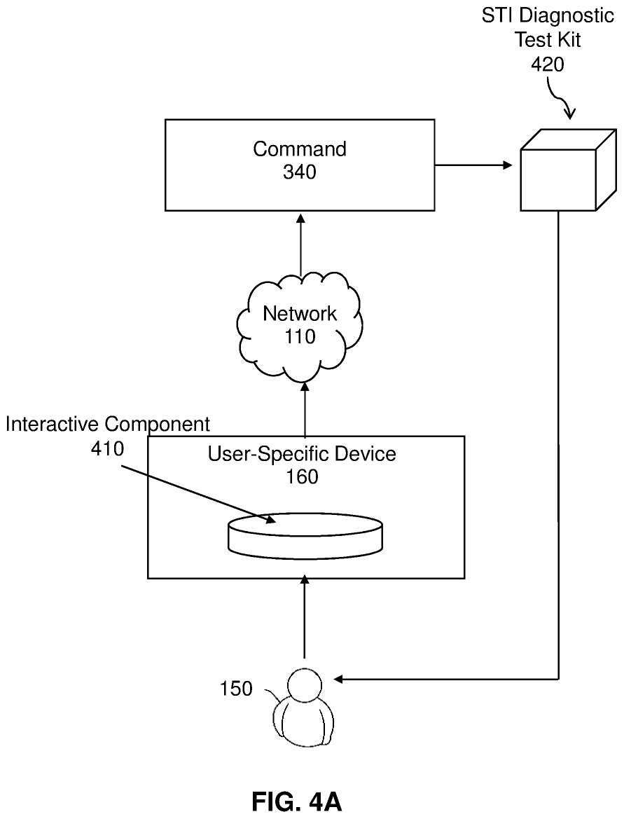

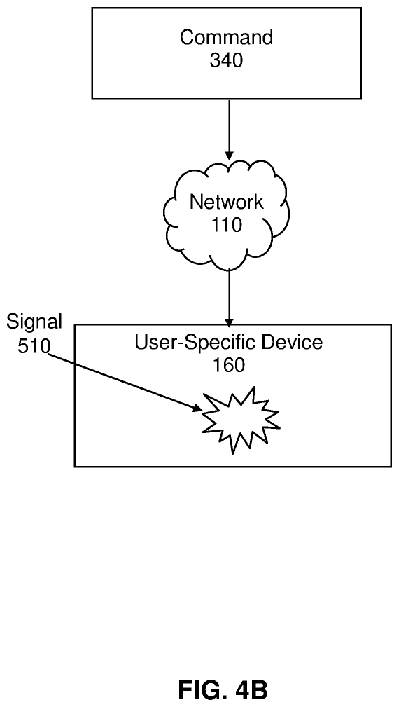

FIG. 4A is a block diagram of an illustrative example of the user 150 interacting with an exemplary user-specific device 160. In this example, the user 150 is requesting an STI diagnostic test kit 420. The user 150 physically interacts with the user-specific device 160 via the interactive component 410 located on the device, which initiates a command 340 previously set by the user 150 in the user health management platform 120 in a user profile 210. In other embodiments of the invention, the interactive component 410 may be a button, switch, touch-screen, or biometric sensor. In an embodiment of the invention, the command 340 is not initiated until the request is authenticated and authorized by the user 150. In this example, the command 340 is to request or order an STI diagnostic test kit 420 to be delivered to the user 150. In some embodiments of the invention, the user 150 may request the diagnostic test, but the request may need to be verified or signed-off by a healthcare provider, such as a physician. As such, the provider may receive an email or some other communication regarding the user 150 request. The physician may be able to access the user health management platform 120 and review, accept and/or deny the request in the user profile 210. As described throughout, this request may include by way of example, ordering a diagnostic test or filling a prescription and the user-specific device 160 may have an interactive component 410 such as at least one of a button, switch, touch-screen, or biometric sensor.

FIG. 4B is another illustrative block diagram of an exemplary user-specific device 160. In this example, a command 340 to provide a prompt was previously set by, for example, the user 150. The prompt may be generated or created based on the user-profile, user controls, or by a communication received from a physician or pharmacy. As described, the prompt may be delivered to the user 150 via the user-specific device 160 and may be in the form of an audio, physical, or visual signal 510 generated by the user-specific device 160. For example, a command may be set by the user 150 to send reminders to the user-specific device 160 to prompt the user 150 to order a diagnostic test. The prompts may be in the form of any type of signal 510, for example a vibration, light, or sound that the user-specific device 160 generates. The reminder or prompt may be to pick-up a prescription, to provide information or status regarding shipment, delivery, or diagnostic testing stage, or that test results are ready. For example, the systems and methods of the invention may also track and report shipment status and delivery status of the diagnostic test kit, status of a returned kit that contains the user's specimen for testing, and the diagnostic testing stage of the sample. In some embodiments, diagnostic testing results are returned to the user and maintained in the user profile. The systems and methods of the invention may use the test results to prompt the user to order another diagnostic test. In other embodiments, a treatment may be recommended or prescribed. In yet another embodiment, the user-specific device 160 may indicate via the signal 510 the status of authorization of the request. The user-specific device may include a power source, for example a battery. The power source may be a rechargeable battery, which may be recharged via a USB, a wall outlet, or any acceptable power source.

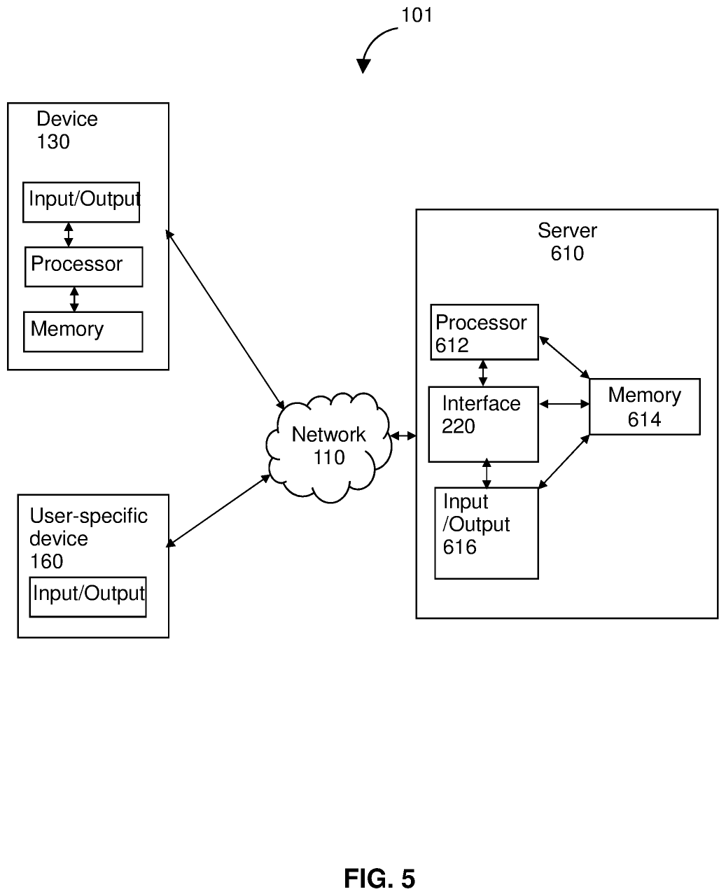

FIG. 5 is a block diagram illustrating at least one embodiment of the system 101 for requesting diagnostic testing services. The system 101 generally includes at least one device 130, such as a mobile device, at least one user-specific device 160, and a server 610. The server includes at least one or more processors, such as processor 612. Processor 612 is operably connected to communication infrastructure such as a communications bus, cross-over bar, or network). The processor 612 may be embodied as any type of processor capable of performing the functions described herein. For example, the processor may be embodied as a single or multi-core processor(s), digital signal processor, microcontroller, or other processor or processing/controlling circuit. In some embodiments, the server is cloud-based.

The server 610 further includes an interface 220 that forwards graphics, text, sounds, and other data from communication infrastructure for display on a device 130 or the user-specific device 160 via the network 110. The server may also include input/output devices 616, which may include interactive components 410 previously described throughout The user-specific device may include the input/output devices 616 for interacting with the user-specific device 160, such as a button, switch, light, vibration, keypad, biometric sensor, microphone, camera, as well as other input components, including motion sensors, and the like. In one embodiment, the user-specific device 160 may include a touch-sensitive display (also known as “touch-screens” or “touchscreens”), in addition to, or as an alternative to, physical push-button keyboard or the like. The touch screen may generally display graphics and text, as well as provides a user interface (e.g., but not limited to graphical user interface (GUI)) through which a user may interact with the user profile via a device 130 or the user-specific device 160, including an app for providing direct user input to the health data management platform.

The server further includes memory 614, such as random access memory (RAM). The memory 614 may be embodied as any type of device or devices configured for short-term or long-term storage of data such as, for example, memory devices and circuits, memory cards, hard disk drives, solid-state drives, or other data storage devices. Similarly, the memory 614 may be embodied as any type of volatile or non-volatile memory or data storage capable of performing the functions described herein.

In the illustrative embodiment, the server 610 may maintain one or more application programs, databases, media and/or other information in the memory 614 that are executable by one or more processors 612. For example, the memory 614 contains the user profile and user commands. In some embodiments, the memory 614 contains at least one module. The module may perform various operations, such as displaying user profile via the interface described herein. A module may also be directed towards creation of user commands to be executed by the processor so as to perform the commands, such as those described herein. The term “module” may refer to any executable instructions, such as software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. Systems and methods of the invention include a module for the collection and management of user data and the module may be in the user health management platform 120 and the module may be configured to communicate and exchange data with the databases described throughout.

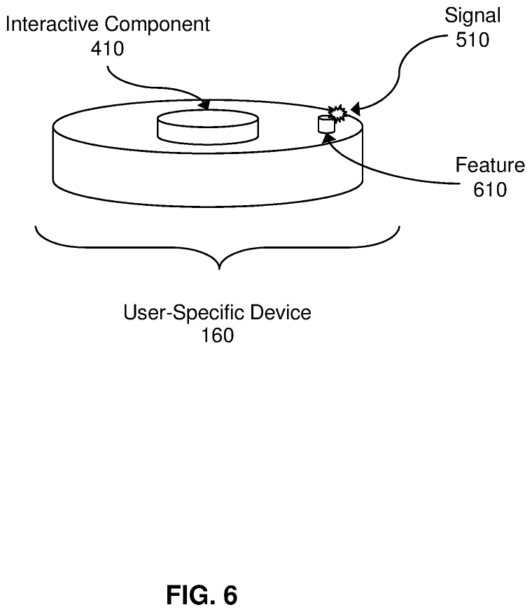

FIG. 6 is a illustrative block diagram of one exemplary embodiment of the user-specific device. In this example, the user-specific device 160 is depicted as being circular and comprises a button 410 that can be pressed to initiate a command for an STI related service. The user-specific device 160 also comprises a feature 610 to generate a signal 510 so as to provide the user with a reminder or prompt previously set.

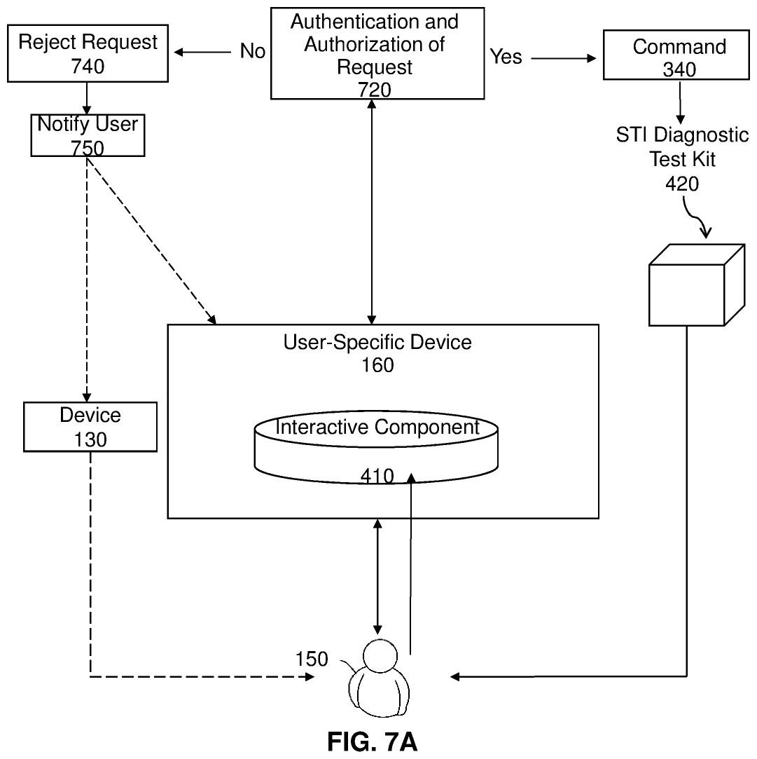

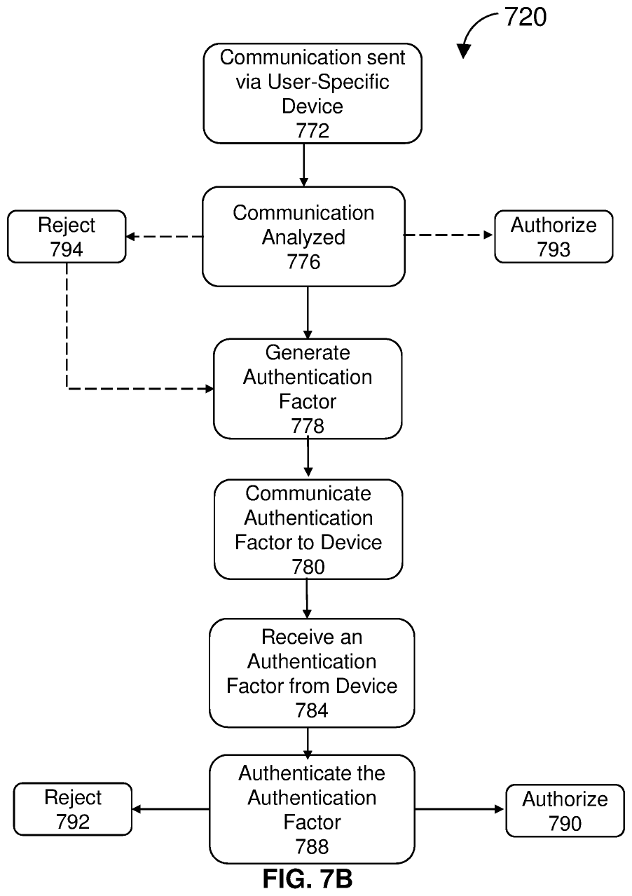

FIG. 7A is a block diagram of an illustrative example of authentication 720 of the user 150 request via the user 150 interacting with an exemplary user-specific device 160. In this example, the user 150 is requesting an STI diagnostic test kit 420. The user 150 physically interacts with the user-specific device 160 via the interactive component 410 located on the device, which initiates authentication 720 performed by a module stored in the memory of the server, before initiating a command 340 previously set by the user 150 in the user health management platform 120 in a user profile 210. The command 340 is not initiated until the request is authenticated and authorized 720 by the user 150. The user-specific device 160 may require the user 150 to enter authentication information via the interactive component 410. A user's authentication information can be a password or a biometric template and can be stored in the memory of the secure server. The systems and methods of the invention authenticate the request by performing one or more of biometric identification, password validation, and multi-factor authentication. The systems and methods of the invention may authenticate the request by comparing the information entered with the request to the authentication information of the user or by multi-factor authentication. A module stored in the memory of the server may contain instructions to perform this function. The instructions are executed by a processor on a server. If authentication and authorization is successful, in this example, the command 340 to request or order an STI diagnostic test kit 420 to be delivered to the user 150 is executed. In other embodiments, the request may not be authenticated and therefore not authorized by the user 150, in such embodiments the request is rejected 740. In such embodiments, the user 150 may receive notification 750 of the rejected request. The notification may be sent via the user-specific device 160 via the interactive component 410 or may be sent to the user's device 130 by email, text message, or via an application of the present invention.

FIG. 7B is a flow diagram of an illustrative example of a process of authentication and authorization 720 of a user's 150 request via a user-specific device 160. A user 150 may interact with the user-specific device 150 via the component 410, which sends a first communication 772. In some embodiments of the invention the first communication 772 is a password or biometric information entered via the user-specific device 160. The first communication 772 is analyzed 776. In some embodiments, analyzing 776 the first communication 772 may include comparing the first communication 772 with the authentication information of the user. In other embodiments, analyzing 776 the first communication 772 is simply acknowledgement of receipt of the first communication 772 for a request. In some embodiments of the invention, once the first communication 772 is analyzed 776, an authentication factor is generated 778 and communicated to the device 130. For example, the authorization factor can comprise a set of one or more random or pseudo-random words, numbers, symbols, images, sounds, or a combination thereof. The authorization factor can be generated by an authorization factor generator, which may generally include any system capable of generating or otherwise accessing an authorization factor. The device 130 may be a mobile device or computer previously registered by the user 150 for future authentication and authorization of requests 720 received from the user-specific device 160. At least a portion of the authentication factor is received from the user's device 130. The at least a portion of the authentication factor is authenticated 788. If authenticated, the request is authorized 790, meaning a command is executed and the request is therefore processed. If authentication fails, the request is not authorized 792, the command is not executed, and the request is therefore not processed.

In an embodiment of the authentication and authorization process 720 where the first communication 772 includes a password or biometric information, analyzing 776 may include comparing the password or biometric information received via the first communication 772 with authentication information stored in the user's profile. In some embodiments, the process of authentication and authorization 720 may be completed upon such comparison. Meaning, if the first communication 776 is authenticated by the comparison, the request is authorized 793. If the comparison fails, authentication fails, and the request is rejected 794. In some embodiments, the rejection 794 prompts the generation of an authentication factor 778, which is communicated to a device 130, 780. At least a portion of the authentication factor is received from the user's device 130. The at least a portion of the authentication factor is authenticated 788. If authenticated, the request is authorized 790, meaning a command is executed and the request is therefore processed. If authentication fails, the request is not authorized 792, the command is not executed, and the request is therefore not processed. Rejection 794 and 792 and authorization 793 and 790, can be communicated to the user 150 via the user-specific device 160 or a device 130.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A system to facilitate services for sexually transmitted infections, the system comprising:
a computing device;
a user-specific device configured to receive a request from a user and wirelessly communicate with the computing device;
a server configured to communicate and exchange data with the computing device, the server comprising a processor coupled to a computer-readable memory containing instructions executable by the processor to cause the server to:
receive input from the computing device to generate a user profile;
associate the user profile with the user-specific device;
receive, from the user-specific device, a request for a sexually transmitted infection (STI) service;
authorize the request; and
process the request.

2. The system of claim 1, wherein the server authorizes the request by authenticating the user via an authentication process selected from the group consisting of: biometric identification, password validation, and multi-factor authentication.

3. The system of claim 2, wherein the server is further configured to receive a command for an STI service from the user, wherein a positive authentication of the user authorizes the command to be executed.

4. The system of claim 3, wherein the command is to order a STI diagnostic test or a therapy.

5. The system of claim 1, wherein the server is further configured to prompt the user to order an STI service or to comply with therapy protocol.

6. The system of claim 5, wherein the user-specific device is further configured to deliver the prompt to the user, and wherein the prompt is an audible or visual stimulus generated by the user-specific device.

7. The system of claim 1, wherein the request is initiated upon the user interacting with the user-specific device.

8. The system of claim 7, wherein the user-specific device comprises an interactive component selected from the group consisting of: a button, a switch, a touch-screen, and a biometric sensor.

9. The system of claim 1, wherein the server is further configured to identify a reoccurring STI service based on the user profile.

10. The system claim 4, wherein the server is further configured to receive and input results from the diagnostic test into the user profile.

11. The system of claim 4, wherein the therapy is an antiviral therapeutic.

12. The system of claim 4, wherein the order is a reoccurring order.

13. The system of claim 4, wherein the prompt is to remind the user to order an antiviral therapy.

14. A computer implemented method, the method comprising:
receiving initial input from a user, wherein the input is used to generate a user profile;
identifying an STI diagnostic test for the user based on the user profile;
receiving a request from the user via a user specific device;
authenticating the request;
ordering the STI diagnostic test kit; and
providing the STI diagnostic test kit to the user, wherein the kit is provided to customer in an unlabeled package.

15. The computer implemented method of claim 14, wherein the STI diagnostic kit comprises a specimen collection device.

16. A method of monitoring compliance with an antiviral therapy protocol, the method comprising:
receiving initial input from a user prescribed an antiviral therapy;
identifying a testing compliance schedule for the user; and
prompting the user to order a compliance test kit via a user-specific device based on the schedule.

17. The method of claim 16, further comprising receiving a request from the user-specific device, wherein the request is generated by the user interacting with the user-specific device.

18. The method of claim 17, further comprising authenticating the user, wherein authentication is selected from the group consisting of: biometric identification, password validation, and multi-factor authentication.

19. The method of claim 18, further comprising authorizing the request and ordering the test, wherein the user is authenticated.

20. The method of claim 19, further comprising re-ordering the therapy, wherein results from the test indicate compliance with the protocol.
